(19) United States
(12) Patent Application Publication
(10) Pub. No.: US 2026/0035653 A1
(43) Pub. Date: Feb. 5, 2026

(54) Automated Cell Culture Systems, Devices, and Associated Methods

(71) Applicants: Emory University, Atlanta, GA (US); Children's Healthcare of Atlanta, Inc., Atlanta, GA (US)

(72) Inventors: Nael McCarty, Atlanta, GA (US); Analia Jazmin Vazquez Cegla, Atlanta, GA (US)

(21) Appl. No.: 19/291,390

(22) Filed: Aug. 5, 2025

Related U.S. Application Data
(60) Provisional application No. 63/679,376, filed on Aug. 5, 2024.

(51) Int. Cl. C12M 1/36; C12M 1/00; C12M 3/00

(52) U.S. Cl. CPC C12M 41/48; C12M 23/38; C12M 23/40; C12M 23/44; C12M 29/00

(57) ABSTRACT

The automated cell culture systems, devices and methods can allow for better control of the chemical and biological environment while doing in vitro cell culture. In some examples, the system may include a main body having a top side, a bottom side, a first side, and a second side. In some examples, the main body may include one or more fluid channels. Each fluid channel may have a first end disposed at the first side and a second end disposed at the second side. Each channel may include an inlet at the first end and an outlet at the second end. Each channel may be defined by opposing side wall surfaces and a bottom surface region. Each inlet may be disposed at a first position along the first side closer to the top side than each outlet. Each bottom surface region may be sloped from the inlet to the outlet.

### CROSS-REFERENCE TO RELATED APPLICATIONS

This application claims the benefit of U.S. Provisional Application No. 63/679,376 filed Aug. 5, 2024. The entirety of this application is hereby incorporated by reference for all purposes.

### BACKGROUND

Cell culture has been a major component of biomedical research and involved in a number of projects focused on understanding mechanisms and treatment methods for diseases. Maintaining cells can present a large financial and time commitment. In addition to this commitment, currently available cell culture techniques generally do not accurately reflect the growing conditions in the body, potentially affecting the conclusions that are drawn during experiments.

### SUMMARY

Thus, there is need for efficient and cost-effective cell culture systems, devices and methods that can be used with automated methods that can allow for better control of the chemical and biological environment while doing in vitro cell culture.

Additional advantages of the disclosure will be set forth in part in the description which follows, and in part will be obvious from the description, or may be learned by practice of the disclosure. The advantages of the disclosure will be realized and attained by means of the elements and combinations particularly pointed out in the appended claims. It is to be understood that both the foregoing general description and the following detailed description are exemplary and explanatory only and are not restrictive of the disclosure, as claimed.

### BRIEF DESCRIPTION OF THE SEVERAL VIEWS OF THE DRAWINGS

The disclosure can be better understood with the reference to the following drawings and description. The components in the figures are not necessarily to scale, emphasis being placed upon illustrating the principles of the disclosure.

FIGS. 1A-1B show views of an assembled culture device according to some embodiments. FIG. 1A shows a view of an assembled device according to some embodiments.

FIG. 1B shows an exploded view of the device of FIG. 1A.

FIGS. 2A-2G show views of the main body of the device shown in FIGS. 1A-1B according to some embodiments. FIG. 2A shows a top view of the main body. FIG. 2B shows a perspective view of the main body. FIG. 2C shows another perspective view of the main body. FIG. 2D shows a rear view of the main body. FIG. 2E shows an expanded, cross-sectional view of the main body. FIG. 2F shows a side view of the main body. FIG. 2G shows another expanded, cross-sectional view of the main body.

FIGS. 3A-3B show views of the tray of the device shown in FIGS. 1A-1B according to some embodiments. FIG. 3A shows a top view of the tray according to some embodiments. FIG. 3B shows a cross-sectional view of the tray shown in FIG. 3A.

FIGS. 4A-4B show views of the lid of the device shown in FIGS. 1A-B according to some embodiments. FIG. 4A shows a top view of the lid according to some embodiments. FIG. 4B shows a side view of the lid shown in FIG. 4A.

FIGS. 5A-5B show views of an example of the partially assembled device with permeable cell culture inserts according to some embodiments. FIG. 5A shows a perspective view of the partially assembled device according to some embodiments. FIG. 5B shows a side view of the partially assembled device shown in FIG. 5A.

FIG. 6 shows an example of an operational flowchart of a cell culture system that can be used to control operation of the culture device according to embodiments.

### DESCRIPTION OF THE EMBODIMENTS

In the following description, numerous specific details are set forth such as examples of specific components, devices, methods, etc., in order to provide a thorough understanding of embodiments of the disclosure. It will be apparent, however, to one skilled in the art that these specific details need not be employed to practice embodiments of the disclosure. In other instances, well-known materials or methods have not been described in detail in order to avoid unnecessarily obscuring embodiments of the disclosure. While the disclosure is susceptible to various modifications and alternative forms, specific embodiments thereof are shown by way of example in the drawings and will herein be described in detail. It should be understood, however, that there is no intent to limit the disclosure to the particular forms disclosed, but on the contrary, the disclosure is to cover all modifications, equivalents, and alternatives falling within the spirit and scope of the disclosure.

The disclosed embodiments relate to systems, devices, and methods that can be used in a programable automated cell culture system capable of efficient and tight control of the chemical and biological environments while doing in vitro cell culture. In some examples, the systems, devices, and methods can be configured for tissue culture and/or biological cell culture. For example, the disclosed embodiments can be used with any number of tissue formats and/or cell formats. By way of example, the formats may include but are not limited to cell monolayers, explants, spheroids, 3D-printed scaffolds, other cell or tissue formats, or any combination thereof.

In some examples, the disclosed cell culture device can allow for reduced media consumption while ensuring good media exchange. Additionally, the disclosed cell culture devices, systems, and methods, can be used in an automated system and do not require bulky and/or specialized equipment.

In some examples, the disclosed cell culture devices can be used to mimic in vitro models to study pathophysiology of a disease, study treatments to a disease, other clinical applications (e.g., timed exposure to small molecules or other potential therapeutic agents), among others, or any combination thereof.

As used herein, the term “medium” or “media” may include any substance that is used in connection with any of the reactions described herein. For example, a media can include a buffer, an enzyme, a cell culture medium, a wash solution, a reagent, therapeutic agent, or the like. A media can include a mixture of one or more constituents. A media can include such constituents regardless of their state of matter (e.g., solid, liquid and/or gas). Moreover, a media can include the multiple constituents that can be included in a substance in a mixed state, in an unmixed state and/or in a partially mixed state. A media can include both active constituents and inert constituents. Accordingly, as used herein, a media can include non-active and/or inert constituents such as, water, colorant, or the like.

In some embodiments, the systems, devices, and methods can be configured for fluid-based transduction protocols. For example, the protocols may include flow transduction (i.e., continuous perfusion), static (e.g., single loading), among others, or a combination thereof.

As used herein, the term “communicate” or “connect” (e.g., a first component “communicates with” or “is in communication with” a second component) or “fluid communication” and grammatical variations thereof are used herein to indicate a fluidic relationship between two or more components and/or channel(s). As such, the fact that one component/channel is said to communicate with a second component/channel is not intended to exclude the possibility that additional components may be present between, and/or operatively associated or engaged with, the first and second components.

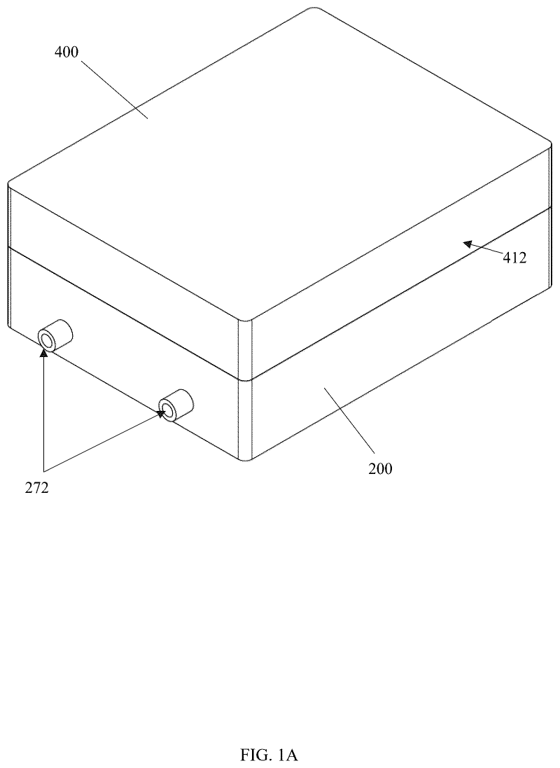

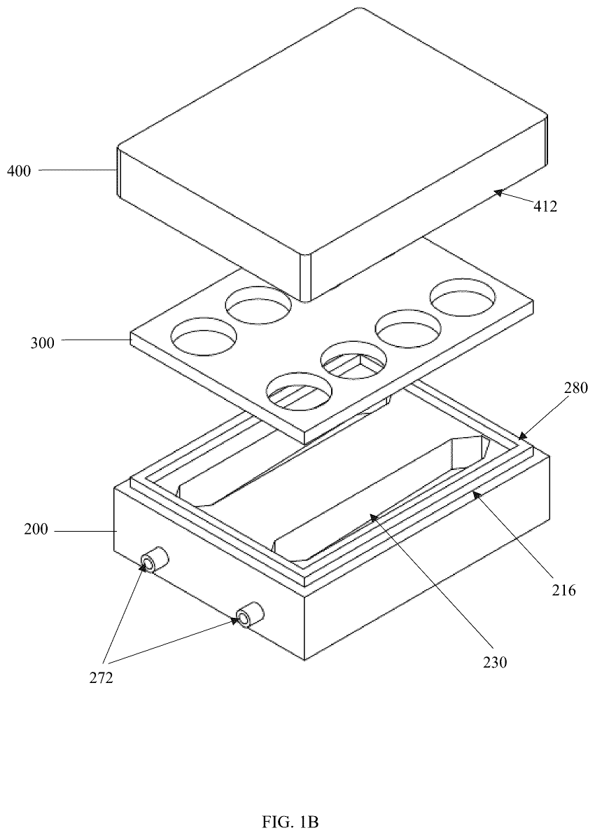

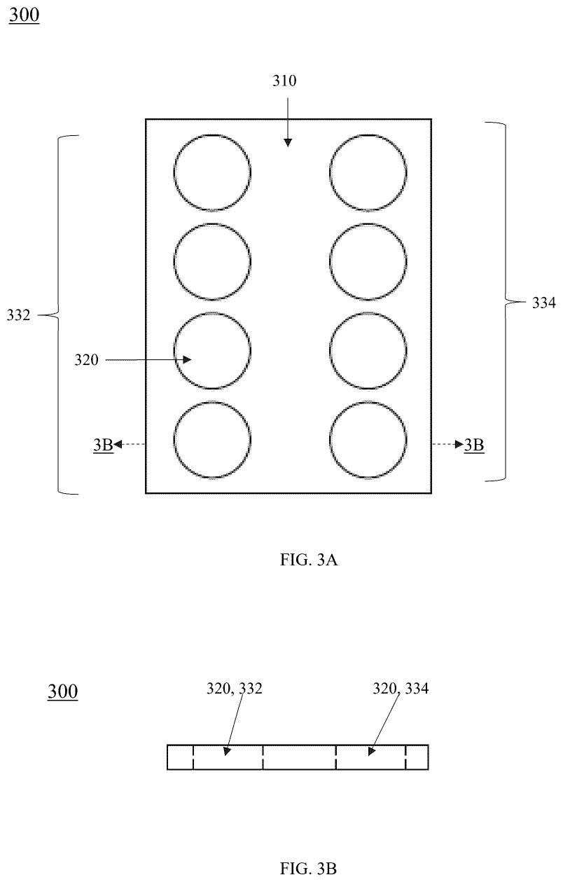

FIGS. 1A-5B show an example of a system and its devices according to some embodiments. FIGS. 1A and 1B show an assembled view and an exploded view of the cell culture device (also referred to as “plate”) 100. As shown in these figures, the device 100 may include a main body 200 having one or more channels 230, a tray 300 configured to be removably disposed on the main body 200, and a lid 400 configured to enclose and cover the tray 300 and channel(s) 230.

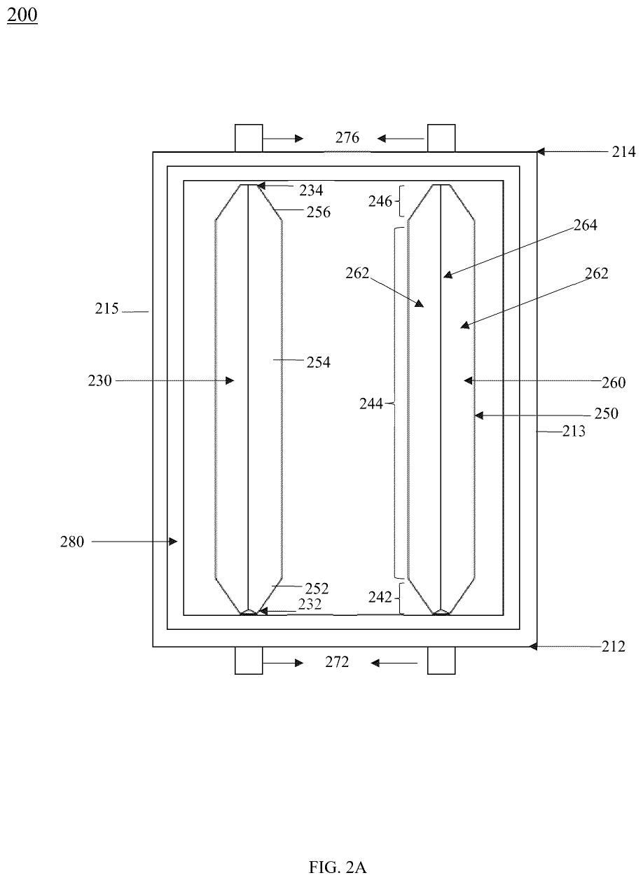

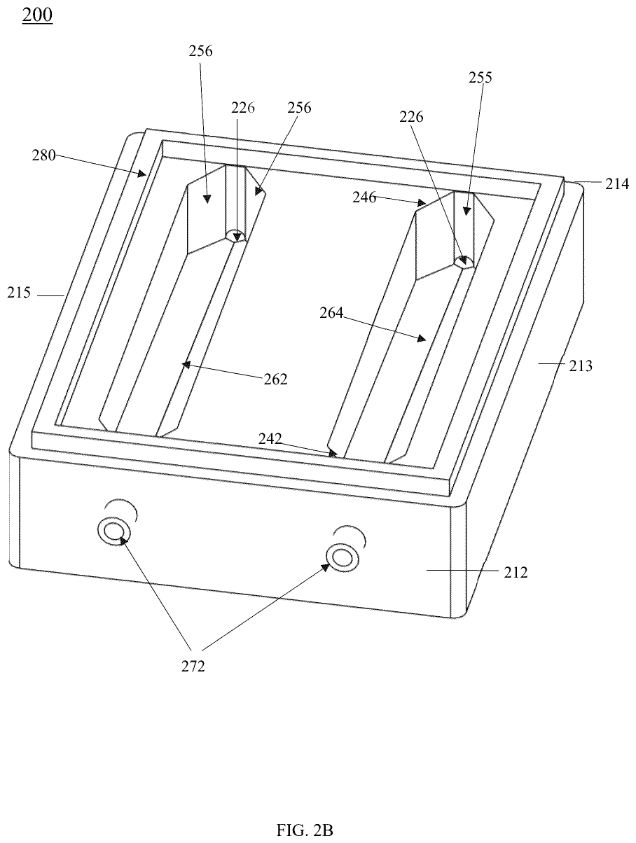

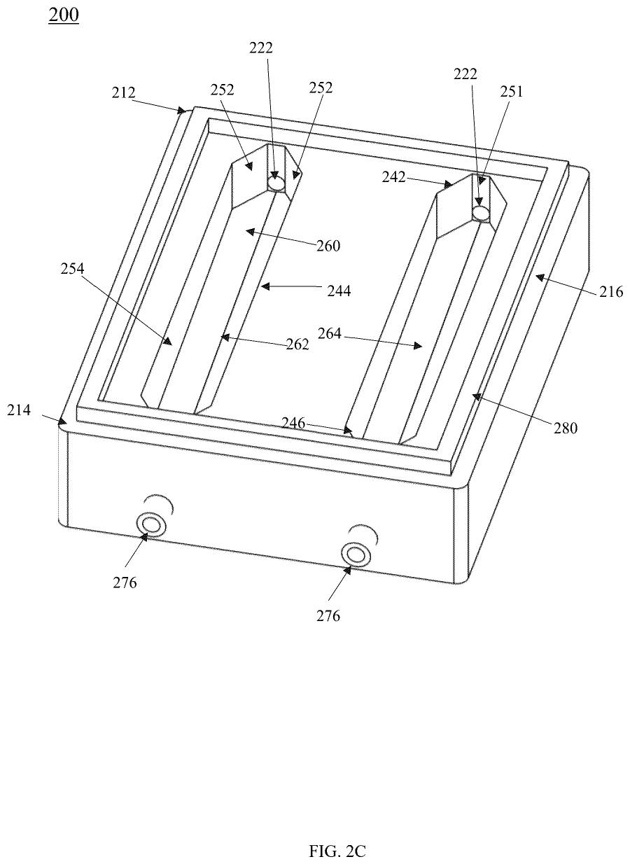

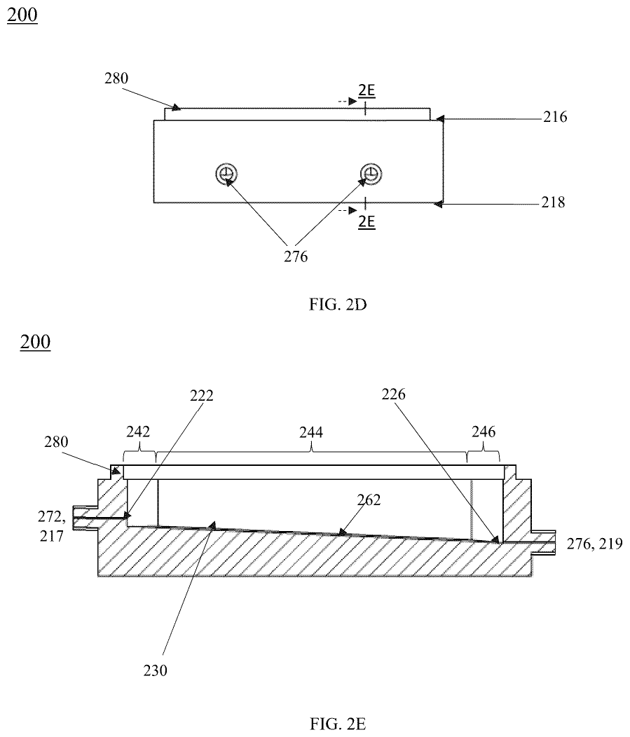

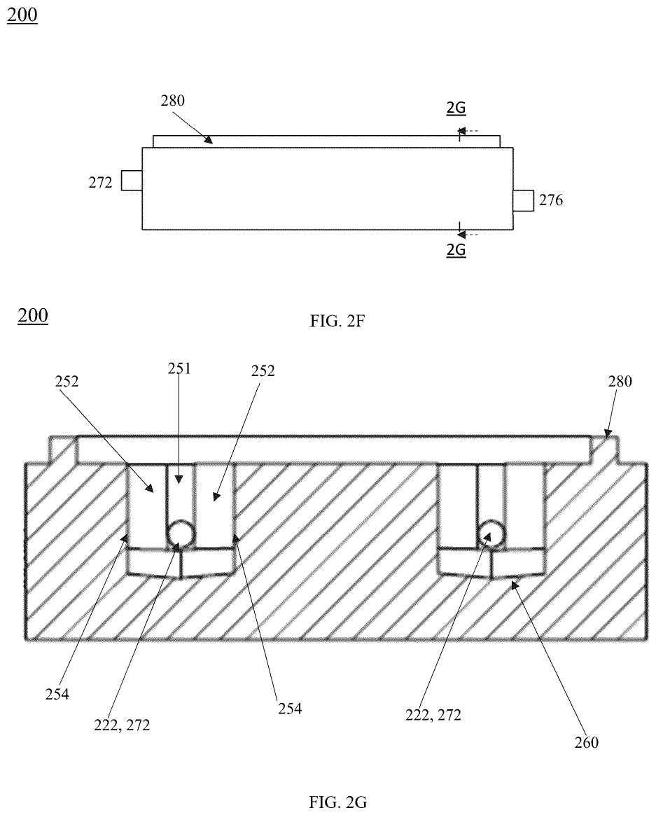

FIGS. 2A-2G show isolated views of the main body 200 according to some embodiments. In some examples, the main body 200 may have a top side 216 and an opposing bottom side 218. In some examples, the main body 200 may have a rectangular shape as shown in the figures. In other examples, the main body 200 may have a different shape. For example, the main body 200 may include a first side 212 extending between the top side 216 and the bottom side 218, and a second side 214 opposing and parallel to the first side 212 and extending between the top side 216 and the bottom side 218. In some examples, the main body 200 may include a first elongated side 213 extending between the first side 212 and the second side 214, and a second elongated side 215 extending between the first side 212 and the second side 214. The first elongated side 213 and the second elongated side 215 being opposing and parallel to each other.

In some examples, the main body 200 may include one or more fluid channels 230 extending between extending between the first side 212 and the second side 214. The one or more fluid channels 230 may extend parallel to the first and second elongated sides 213, 215. Each channel may be considered a separate cell culture reservoir. This way, different genotypes and/or treatments can be studied in parallel (in respective channel 230 of the device 100) without the possibility of cross-contamination between cell lines.

In some examples, the main body 200 may include two channels 230 as shown in the figures. In other examples, the main body 200 may include any number of channels 230. In some examples, the two or more channels 230 may have the same dimensions as shown in the figures. In other examples, the two or more channels 230 may have different dimensions (e.g., length, width, cross-section, etc.).

In some examples, each fluid channel 230 may have a first end 232 disposed at the first side 212, a second end 234 disposed at the second side 214, and a length therebetween. In some examples, each channel 230 may include an inlet 222 at the first end 212 and an outlet 226 at the second end 214.

In some examples, the main body 200 may include an adapter on the external surface of the main body 200 that is in fluid communication and corresponding to location of each inlet 222 and each outlet 226. For example, the main body 200 may include an adapter 272 disposed externally on the first side 212 that is in fluid communication and in line with the inlet 222 and an adapter 276 disposed externally on the second side 214 that is in fluid communication and in line with the outlet 226. In some examples, the adapters 272, 276 may be any fluid connectors, such as tubing, valves, luer locks, among others, or any combination thereof.

In some examples, each channel 230 may be an exposed, open fluid channel. For example, each channel 230 may be defined by opposing side wall surfaces 250 and a bottom surface region 260 disposed between the opposing side wall surfaces 250.

In some examples, each inlet 222 and each outlet 226, and respective adapters 272 and 276, may be disposed at different positions on the respective sides with respect to the top and bottom sides 216, 218 (i.e., height defined by the length between the top side 216 and the bottom side 218). For example, as shown in the figures, each inlet 222 and its adapter 272 may be disposed at a first position 217 along the first side 212 and each respective outlet 226 and its adapter 276 may be disposed at a second position 219 along the second side 214. In some examples the inlet 222 and its adapter 226 may be closer to the top side 216 than each respective outlet 226 and its adapter 272 so that each respective outlet 226 and its adapter 272 is disposed along the second side 214 closer to the bottom side 218.

In some examples, each bottom surface region 260 may be sloped from the respective inlet 222 to the respective outlet 226 so that the cross-sectional volume of the channel 230 is greater towards the outlet 226 of the respective channel 230. As shown, each bottom surface region 260 may have a gradual decline along the length from the respective inlet 222 to the respective outlet 226 with respect to the sides 212, 214. This can help drive fluid flow from the inlet 222 to the respective outlet 226.

In some examples, the height of opposing side wall surfaces 250 may result in the gradual decline of the bottom surface region 260. For example, the opposing side wall surfaces 250 may increase in height (e.g., defined by the length between the bottom surface region 260 and the top side 216) along the length of the channel 230 from the inlet 222 towards each outlet 226. As shown in the figures, the height of the wall surfaces 250 at the inlet 222 may be the smallest and the height of the wall surfaces 250 at the respective outlet 226 may be the largest.

In some examples, each bottom surface region 260 may have a concave shape. For example, each bottom surface region 260 includes a center axis 262 and opposing bottom wall surfaces 264 that extend at an angle between the center axis 262 and the respective wall surface 250. In some examples, the center axis 262 may be closest to the bottom side 218 of the main body 200.

In some examples, widths of each bottom wall surface 264 may be the same along the length of the channel 230 (from the inlet 222 to the outlet 226). For example, the widths of each bottom wall surface 264 may be defined by the length of the wall surface 260 between the center axis 262 and the respective wall surface 250. This way, the center axis 262 may extend between the inlet 222 and the outlet 226 in a gradual decline due to the tapered height of the wall surfaces 250.

In some examples, each channel 230 may include one or more sections disposed along the length. For example, the one or more sections may include an entry section 242 disposed at the inlet 222, an exit section 246 disposed at the outlet 226, and a main section 244 disposed between the entry section 242 and the exit section 246.

In some examples, the opposing wall surfaces 250 may taper inward in the entry section 242 with respect to the inlet 222 and taper inward in the exit section 246 with respect to the outlet 226. For example, as shown in FIGS. 2B and 2C, the opposing wall surfaces 254 of the main section 264 may be disposed in parallel and may increase in height along the length from the entry region 242 toward the exist section 246. As shown in FIG. 2C, each entry region 242 may include two opposing wall surfaces 252 that taper from the respective wall surfaces 254 to an inlet wall surface 251 that is at the first side 212. As shown, the inlet 222 may be disposed on the inlet wall surface 251 and in fluid communication with the adapter 272.

As shown in FIG. 2B, each exit region 246 may include two opposing wall surfaces 256 that taper from the respective wall surfaces 254 to an outlet wall surface 255 that is at the second side 214. As shown, the outlet 226 may be disposed on the outlet wall surface 255 and in fluid communication with the adapter 276.

In this example, the height of the wall surfaces 254 and 255 of the exit section 246 may be larger than the height of the wall surfaces 252 and 251 of the entry section 242.

In some examples, as shown in the figures, the main body 200 may include a protruding member 280 disposed on the top side 216 of the main body 200. The protruding member 280 may be disposed along the border of the main body 200 and configured to receive the tray 300.

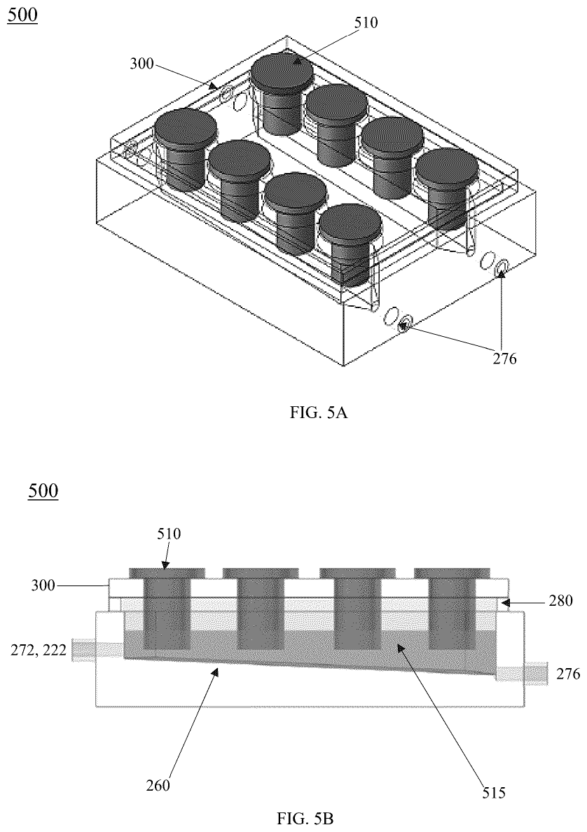

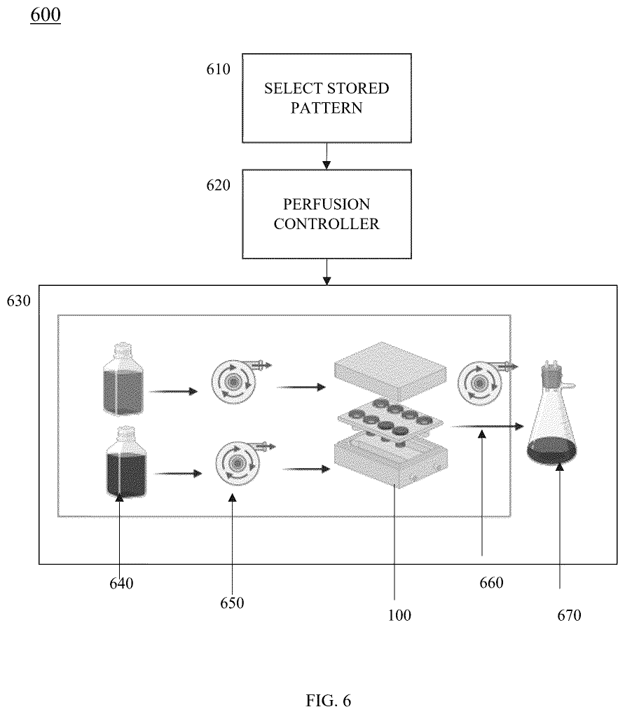

In some examples, the tray 300 may include one or more groups of openings 320 configured to receive a permeable culture insert 510, for example, as shown in an example 500 shown in FIGS. 5A and 5B. For example, the permeable culture inserts 510 may be a Transwell insert. In some examples, the number of groups of openings 320 may correspond to the number of channels of the main body 200. In this example, tray 320 may include a first group 332 and a second group 334 of openings 320 to correspond to a respective channel. The openings 320 of the tray 300 may be disposed so as to be within the main section 244 of the main body 200 when tray 300 is disposed on the protruding member 280.

In some examples, as shown FIGS. 5A and 5B, when a permeable culture insert 540 is disposed in the tray 300 and the tray 300 is disposed on the protruding surface 280 of the main body 200, a bottom of each permeable well insert 510 may be aligned with the inlet 222 of the respective channel 230 so that a media 515 would cover a portion of the insert 510.

In this example, the number and dimensions of the openings 320 are the same in each group 332 and 334. It will be understood that the number of openings 320 and/or dimensions can differ within and/or between each group 332, 334 of openings 320.

In some examples, the device 100 may be made of one or more materials capable of being autoclaved for easy repeated use. For example, the one or more materials may include but is not limited to medical grade resin.

It will be understood that there may be alternative and/or additional inlets, outlets, channels, openings, and the like. For example, the number of channels and/or the features (e.g., inlets, outlets, additional ports, etc.) of the main body, the number of groups and/or the number of openings and/or the features (e.g., size of openings), and/or the like discussed therein can be tailored to suit the numbers of cells to be analyzed and/or analyses to be performed.

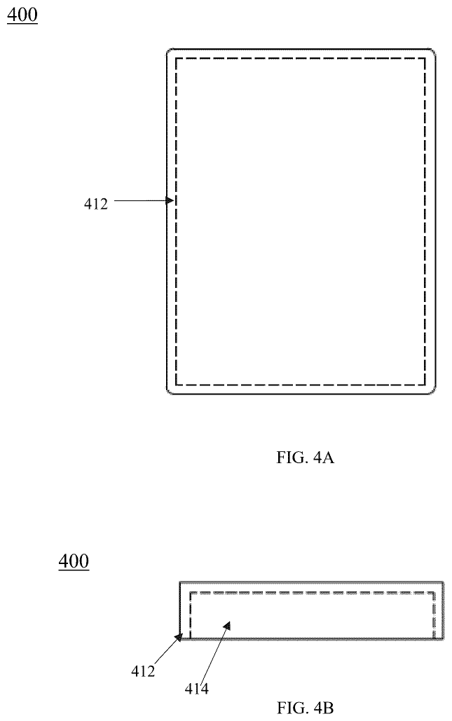

In some examples, the lid 400 may be configured to be removably coupled to the main body 230 and configured to enclose the tray 300 and the one or more channels 230 of the main body 200 as shown in FIGS. 1A and 1B. As shown in FIGS. 4A and 4B, the lid 400 may include a border surface 412 that is configured to be disposed on the top side 216 of the main body 200. In some examples, the lid 400 may include a space 414 defined by the border surface 412 and in which the tray and permeable well inserts 510 may be disposed with clearance. This way, the lid 400 can prevent contamination.

In some examples, the device 100 may be a part of an automated, cell culture system. In some examples, the cell culture system may further include one or more programable perfusion controllers configured to be configured to control the fluid flow of a media from one or more media reservoirs through the respective channel 230 by controlling one or more pumps; the one or more pumps (e.g., peristaltic pump) configured to be controlled by the one or more programable perfusion controllers and in fluid communication with the respective inlet via the adapter 272 and the one or more media reservoirs; the one or more media reservoirs configured to the media and in fluid communication with the one or more pumps and the respective channel 230; one or more suction devices (e.g., pinch valve) configured to be attached to the outlet 226 via the adapter 276 and configured to remove the waste to one or more waste reservoirs in fluid communication with the respective suction device and channel 230; the one or more waste reservoirs configured to receive and store waste media; among others; or any combination thereof. In some examples, at least the media reservoir(s), pump(s), suction device(s), waste reservoir(s), and associated culture device may be configured to be stored inside a cell culture incubator.

In some examples, the system may have perfusion controller(s), media reservoir(s), pump(s), suction device(s), and/or waste reservoir(s) specific for each channel 230 so that the delivery and/or removal of media for each channel 230 may be individually controlled. In some examples, the system may have perfusion controller(s), media reservoir(s), pump(s), suction device(s), and/or waste reservoir(s) specific for more than one channel 230 so that the delivery and/or removal of media may be controlled for more than one channel 230 simultaneously. For example, one perfusion controller may be configured to control more than one pump that is in fluid communication with one or more channels.

In some examples, the one or more programmable perfusion controllers may be configured to control delivery of media according to one or more stored perfusion patterns, user-inputted perfusion pattern, among others, or any combination thereof. In some examples, the one or more perfusion patterns may mimic in vivo physiologically relevant conditions. For example, the one or more perfusion patterns may include but is not limited to patterns that mimic meal-like glucose fluctuations. By way of example, Cystic Fibrosis-Related Diabetes may be studied by controlling the delivery of glucose media according to a meal-like glucose fluctuation pattern to a culture device having immortalized and primary airway epithelial cells expressing either wild-type (WT) or mutant Cystic Fibrosis Transmembrane Conductance Regulator (CFTR) cultured in the one or more channels.

FIG. 6 shows an example 600 of operation of a cell culture system according to embodiments. In some examples, the user may first select the perfusion pattern 610. In some examples, the user may select the perfusion pattern from a plurality of stored patterns. In other examples, the user may program the desired perfusion pattern. Next, the programable perfusion controller 620 may control the fluid flow of media from media reservoirs 640 through the device 100 according to the selected perfusion pattern 610. For example, the controller 620 may control (i) the pumps 650 that are in fluid connection with the media reservoirs 640 and the device 100 (ii) and the pinch valve(s) s 660 that are in fluid connection with the waste reservoir 670 and the device 100.

While the disclosure has been described in detail with reference to exemplary embodiments, those skilled in the art will appreciate that various modifications and substitutions may be made thereto without departing from the spirit and scope of the disclosure as set forth in the appended claims. For example, elements and/or features of different exemplary embodiments may be combined with each other and/or substituted for each other within the scope of this disclosure and appended claims.

What is claimed:

1. A cell culture system, comprising:
a main body having a top side, an opposing bottom side, a first side extending between the top side and the bottom side, and a second side opposing the first side and extending between the top side and the bottom side;
the main body including one or more fluid channels extending between the first side and the second side;
each fluid channel having a first end disposed at the first side, a second end disposed at the second side, and a length therebetween;
each channel including an inlet at the first end and an outlet at the second end;
each channel being defined by opposing side wall surfaces and a bottom surface region disposed between the opposing side wall surfaces;
each inlet being disposed at a first position along the first side closer to the top side than each outlet so that each outlet is disposed along the side second closer to the bottom side; and
each bottom surface region being sloped from the respective inlet to the respective outlet so that the cross-sectional volume of the channel is greater towards the outlet of the respective channel.

2. The system of claim 1, wherein:
each bottom surface region includes a center axis and opposing bottom wall surfaces that extend from the center axis.

3. The system of claim 2, wherein:
each bottom wall surface has a width between the center axis and the respective side wall surface; and
the widths of the opposing bottom wall surfaces are the same.

4. The system of claim 3, wherein:
each opposing side wall surface has a height disposed between the bottom surface region and the top side; and
the height of each opposing side wall surface increases from the inlet to the outlet of each channel.

5. The system of claim 3, wherein:
each channel includes one or more sections disposed along the length;
the one or more section includes an entry section, an exit section, and a main section disposed between the entry section and the exit section;
the entry section disposed at the inlet;
the exit section disposed at the outlet; and
the opposing wall surfaces of the main section are disposed in parallel.

6. The system of claim 5, wherein the opposing wall surfaces of the entry section are tapered inward from the main section toward the inlet.

7. The system of claim 6, wherein the opposing wall surfaces of the exit section are tapered inward from the main section toward the outlet.

8. The system of claim 1, wherein:
each channel includes one or more sections disposed along the length;
the one or more section includes an entry section, an exit section, and a main section disposed between the entry section and the exit section;
the entry section disposed at the inlet;
the exit section disposed at the outlet; and
the opposing wall surfaces of the main section being in parallel.

9. The system of claim 8, wherein the opposing wall surfaces of the entry section are tapered inward from the main section toward the inlet.

10. The system of claim 9, wherein the opposing wall surfaces of the exit section are tapered inward from the main section toward the outlet.

11. The system of claim 1, further comprising:
a tray being configured to be removably coupled to the top side of the main body; and
the tray including one or more groups of one or more openings configured to receive a permeable culture insert, each group being disposed to correspond to a channel of the main body.

12. The system of claim 11, wherein the main body includes two or more channels and the tray includes two or more groups of openings.

13. The system of claim 11, wherein:
the main body further includes a protruding member disposed on the top side along a border of the main body; and
the tray is configured to be disposed on the protruding member.

14. The system of claim 13, further comprising:
one or more permeable culture inserts disposed in the openings of the tray;
wherein when each permeable culture insert is disposed in the tray and the tray is disposed on the protruding surface of the main body, a bottom of each permeable well insert is aligned with the inlet of the respective channel.

15. The system of claim 13, further comprising:
a lid being configured to be removably coupled to the main body and configured to enclose the tray and the one or more channels of the main body.

16. The system of claim 1, further comprising:
an adapter disposed at each inlet, the adapter of each inlet configured to connect to a pump to deliver a media to the respective channel.

17. The system of claim 16, further comprising:
an adapter disposed at each outlet, the adapter of each outlet being configured to connect to a vacuum to suction the media out of the respective channel.
